SEQUENCE LISTING (9 sequences)

SEQ ID NO: 1 - DNA, 3357 bp, Artificial
  Expression cassette
gaattctacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaacacgtatttttacaacaataccaacaacaacaacaacaaacaacattacaattacgtatttctctctctagaatggccaccattgcgttctcccggctgtccatctacttctgcgtgctgctgctgtgccacggctccatggccgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccagtgagagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattc

SEQ ID NO: 2 - DNA, 1448 bp, Oryza sativa
tacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaactcacatcaaccagcccaa

SEQ ID NO: 3 - DNA, 73 bp, Artificial
  LLTCK Leader sequence
acacgtatttttacaacaataccaacaacaacaacaacaaacaacattacaattacgtatttctctctctaga

SEQ ID NO: 4 - DNA, 72 bp, Artificial
  Nucleotide sequence encoding SPGluB4
atggccaccattgcgttctcccggctgtccatctacttctgcgtgctgctgctgtgccacggctccatggcc

SEQ ID NO: 5 - DNA, 1494 bp, Artificial
  Nucleotide sequence encoding the mature form of human acid beta-glucosidase
gcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccagtga

SEQ ID NO: 6 - DNA, 253 bp, Agrobacterium tumefaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 7 - DNA, 30 bp, Artificial
  Artificially synthesized primer sequence
gcatgcgaattctacagggttccttgcgtg

SEQ ID NO: 8 - DNA, 30 bp, Artificial
  Artificially synthesized primer sequence
tctagaagctatttgaggatgttattggaa

SEQ ID NO: 9 - DNA, 2850 bp, Artificial
  Artificially synthesized GAA sequence
atggccaccattgcgttctcccggctgtccatctacttctgcgtgctgctgctgtgccacggctccatggccggccacatcctgctgcatgacttcctgctggtgcctcgggagctgtccggctccagcccggtgctggaggagacccacccggcgcaccagcagggcgcgtcccggccgggccctcgggatgcgcaggcgcacccgggccggcctcgggccgtgcccacccagtgcgatgtgcctcccaactcccggttcgactgcgcgccggacaaagccatcacccaggagcagtgcgaggcgcgtggctgctgctacatcccggccaagcagggcctgcagggcgcgcagatgggccagccctggtgcttcttccctccctcctatccctcctacaagctggagaacctgtcctcctccgagatgggctacaccgccaccctcacccggaccacgcccaccttctttcccaaagacatcctcaccctgcggctggatgtgatgatggagaccgagaaccggctgcatttcaccatcaaagacccggccaaccggcggtatgaggtgcctctggagacgcctcgggtgcattcccgggcgcccagccctctgtactccgttgagttctccgaggagcccttcggcgtgattgtgcggcggcagctggatggccgggtgctgctcaacaccaccgttgcgcctctgttcttcgccgaccagttcctgcagctgtccacctccctgccctcccagtacatcaccggcctggccgagcacctgagccctctcatgctgtccacctcctggacccggatcaccctgtggaaccgggacctggcgcccacgccgggcgccaacctgtatggctcccatcccttctacctggcgctggaggatggcggatccgcgcatggcgtgttcctgctcaactccaatgccatggatgttgtgctgcagcccagcccggcgctgtcctggcggtccaccggcggcatcctggatgtgtacatcttcctgggcccggagccgaagtccgttgtgcagcagtacctggatgttgttggctatcccttcatgcctccctactggggcctgggcttccacctgtgccggtggggctactcctccaccgccatcacccggcaggttgttgagaacatgacccgggcgcatttccctctggatgtgcagtggaatgacctggactacatggactcccggcgggacttcaccttcaacaaagatggcttccgggacttcccggccatggtgcaggagctgcaccagggcggccggcggtacatgatgattgttgacccggccatctcctcctccggcccggccggctcctaccggccctatgatgagggcctgcggcgtggcgtgttcatcaccaatgagaccggccagcctctcattggcaaagtgtggccgggctccaccgcgttcccggacttcaccaatcccaccgcgctggcgtggtgggaggacatggttgccgagttccatgaccaggtgcccttcgatggcatgtggattgacatgaatgagccctccaacttcatccgtggctccgaggatggctgtcccaacaatgagctggagaaccctccctatgtgccgggcgttgttggcggcaccctgcaggccgccaccatctgcgcgtcctcccaccagttcctgtccacccattacaacctgcacaacctgtatggcctcaccgaggccattgcgtcccaccgggcgctggtgaaagcgcgtggcacccggcccttcgtgatctcccggtccaccttcgccggccatggccggtatgccggccattggaccggcgatgtgtggtcctcctgggagcagctggcgtcctcggtaccggagatcctgcagttcaacctgctgggcgtgcctctggttggcgccgatgtgtgcggcttcctgggcaacacctccgaggagctgtgcgtgcggtggacccagctgggcgcgttctatcccttcatgcggaaccacaactccctgctgtccctgcctcaggagccctactccttctccgagccggcgcagcaggccatgcggaaggcgctcaccctgcggtacgcgctgctgcctcacctgtacaccctgttccaccaggcgcatgttgccggcgagaccgttgcgcggcctctgttcctggagtttcccaaagactcctccacctggaccgttgaccaccagctgctgtggggcgaggcgctgctcatcacgccggtgctgcaggccggcaaagccgaggtgaccggctacttccctctgggcacctggtatgacctgcagaccgtgcccattgaggcgctgggctccctgcctcctcctccggccgcgcctcgggagccggccatccattccgagggccagtgggtgaccctgccggcgcctctggacaccatcaatgtgcacctgcgggccggctacatcatccctctgcagggcccgggcctcaccaccaccgagtcccggcagcagcccatggcgctggccgttgcgctcaccaaaggcggcgaggcgcgtggcgagctgttctgggatgatggcgagtccctggaggtgctggagcgtggcgcgtacacccaggtgatcttcctggcgcggaacaacaccattgtgaatgagctggtccgggtgacctccgagggcgccggcctgcagctgcagaaagtgaccgtgctgggcgttgccaccgcgcctcagcaggtgctgtccaatggcgtgccggtgtccaacttcacctacagcccggacaccaaagtgctggacatctgcgtgtccctgctcatgggcgagcagttcctggtgtcctggtgctag

(19) United States
(12) Patent Application Publication
(10) Pub. No.: US 2011/0038971 A1
(43) Pub. Date: Feb. 17, 2011

(54) Method for the Production of Human Recombinant Lysosomal Enzymes in a Cereal Endosperm

(75) Inventors: Stefano Marchetti, Pagnacco (IT); Bruno Bembi, Trieste (IT); Tamara Patti, Buja (IT); Piero Cristin, Gorizia (IT)

Correspondence Address: FLASTER/GREENBERG P.C.;Four Penn Center, 1600 John F. Kennedy Boulevard, 2nd Floor, PHILADELPHIA, PA, 19103, US

(73) Assignee: Transactiva SrL, Udine (IT)

(21) Appl. No.: 12/922,292

(22) PCT Filed: Mar. 11, 2009
(86) PCT No.: PCT/EP2009/052832

§ 371 Date: Sep. 13, 2010

(30) Foreign Application Priority Data
Mar. 13, 2008 (IT) UD2008A000055

(51) Int. Cl. A01N 65/00; A01H 1/00; C12N 9/14; C07H 21/04; C12N 15/82; C12N 1/21; C12N 5/10; A01H 5/00

(52) U.S. Cl. 424/776; 800/288; 435/195; 536/23.2; 435/320.1; 435/252.33; 435/252.3; 435/419; 435/412; 800/298; 800/320.2

(57) ABSTRACT

A method for the production of a human recombinant lysosomal enzyme in a cereal plant endosperm, comprising:
a first step of cereal plant transformation whereby the lysosomal enzyme is obtained and confined in an endosperm, which is not eventually absorbed by the embryo, and the presence of large quantities of the lysosomal enzyme in the endosperm does not negatively affect seed viability and germination speed;
the use, in the first step of cereal plant transformation, of an endosperm-specific promoter upstream the gene encoding said lysosomal enzyme, and of a signal peptide for a co-translational transfer of the newly synthesized lysosomal enzyme into the lumen of the endoplasmic reticulum of the endosperm cells for its tissue-specific accumulation;
a second step of lysosomal enzyme accumulation inside the seed endosperm of a cereal plant.

### BACKGROUND OF THE INVENTION

1. Field of the Invention

The present invention relates to the production of recombinant human lysosomal enzymes, in particular of acid beta-glucosidase (E.C. 3.2.1.45) and acid alpha-glucosidase (E.C. 3.2.1.20), by transformation and genetic manipulation of plants, namely cereal species. The species this invention is preferentially applied to is Oryza sativa L. (cultivated rice) because industrial seed manufacturing can be performed with removal of germ and aleuronic layer, i.e. seed parts containing most lipid and protein contaminants.

The same technology can be applied for the endosperm-specific expression of other human lysosomal enzymes whose deficit or incomplete functionality causes pathological conditions.

2. Description of Related Art

Rare diseases represent a heterogeneous group of disorders which have a low incidence and prevalence in the population.

They show a chronic course and may have severe invalidating consequences or be fatal.

Rare diseases include lysosomal storage disorders, which are caused by the deficit of specific lysosomal enzymes or carrier proteins. This class of disorders comprises, among others, Gaucher disease, Glycogenosis type II, Fabry disease, Niemann-Pick B disease, Mucopolysaccharidoses I, II, IV. The therapeutic approach for these diseases consists in the intravenous administration of the missing enzyme (enzyme replacement therapy, ERT). For example, Gaucher disease can be treated by regular lifelong infusions of human acid beta-glucosidase. However, this therapy is very expensive and thus it is not accessible to all patients. The high cost of ERT is substantially determined by difficulties in acid beta-glucosidase production by means of cultured human or mammalian cells.

Lysosomal hydrolases are difficult enzymes to produce and this for many causes. Firstly, they are needed in low amounts within cells; for energy-saving reasons, their over-expression is prevented by regulatory processes based upon feed-back mechanisms. Regulatory processes are remarkably conserved among species and hinder the achievement of significant production rates of recombinant lysosomal enzymes in the genetically transformed hosts. A well-known example is the production of human beta-glucosidase in animal cells where the TCP80 protein efficiently inhibits the translation of the human beta-glucosidase messenger RNA by altering its binding to polysomes.

Secondly, lysosomal enzymes have a catabolic function over a range of important cellular components and may cause cell damage or death if not correctly displaced in the host cells. In nature, these enzymes are synthesised as inactive precursors that move to lysosomes where they are converted into the corresponding active forms maintained thereafter under strict confinement. In cultured animal cells, recombinant lysosomal enzymes can be allocated in lysosomes or secreted in the culture medium; both solutions are nevertheless suboptimal in terms of production rates or cost. In plants, the choice of the allocation site for recombinant lysosomal enzymes is highly uncertain because plant cells have no lysosomes. On the other hand, misplacing lysosomal enzymes in a plant cell is extremely detrimental in terms of cell viability or metabolic behaviour; in fact, these enzymes can exert their catabolic function over essential cell components, e.g. beta-glucosidase can cleave glycolipids which are constituents of the cell membrane system.

Once the problem of phytotoxicity is adequately solved, genetically engineered plants could represent an alternative production system for lysosomal enzymes, from both a technological and economic point of view, since plant cultivation requires relatively inexpensive materials and agricultural infrastructures that already exist in the territory.

In WO-A-97/10353 (WO'353), the synthesis of lysosomal enzymes, comprising human acid beta-glucosidase and its mutated forms, is reported exclusively in the leaf and, in particular, in the leaf of a biomass species such as tobacco (Nicotiana tabacum L.). WO'353 describes a problematic method in which the high water content of leaf tissues (meaning a high dispersion of the protein of interest) and the presence of a great number of protein contaminants, polyphenols, rubbers, exudates, toxic alkaloids, contribute to complicate the processes of enzyme extraction and purification.

In WO'353, the expression of acid beta-glucosidase follows tobacco transformation with expression vectors harbouring the MeGa promoter (wound-inducible, derived from tomato HMG2 promoter) or the CaMV35S promoter. The latter is a well-known, widely-used element for gene transcriptional control of constitutive type. In WO'353, it is stated that other constitutive or inducible promoters can be used for the same purpose.

As far as wound-inducible promoters are concerned, in WO'353 their use is strictly confined to the leaf, and plants must be previously wounded in order to express acid beta-glucosidase. Preliminary wounding determines an increase in costs, a more complex management of the production process and, in all likelihood, a partial enzyme degradation by proteinases normally resident in the vacuole or in other cellular compartments, as well as a heavier contamination of the wounded material with bacteria and fungi.

Light-inducible promoters virtually considered in WO'353 are not effectively expressed in tissues other than the leaf mesophyll, such as the seeds and particularly plant cereal seeds, due to the lack of transmitted light radiation and/or the lack of transcriptional factors normally present in photosynthetic tissues.

With respect to constitutive promoters, all examples are referred to the CaMV35S promoter, also in view of its representativeness. However, experiments have shown that the CaMV35S transcriptional efficiency is marginally low in the seed, insomuch as to exclude any interest in the synthesis of heterologous proteins. This assertion is even more valuable in the case of monocot plants such as cereals, where this promoter appears less suitable to direct high gene expression levels in the leaf tissues too.

In WO-A-03/073839 (WO'839), it has been reported that a gene coding for a mutated form of beta-glucosidase under the control of CaMV35S promoter is not expressed in the seed at levels detectable in immunological assays carried out with a specific antibody.

Therefore, along with what is asserted in WO'839, it is possible to conclude that patent WO'353 does not actually provide the teachings to perform the production of human acid beta-glucosidase or other lysosomal enzymes in tissues different from the leaf mesophyll and specifically in the seed.

Furthermore, later experiments (Reggi et al. 2005, Plant Mol Biol 57: 101-113) have demonstrated that the information given in WO'353 as to beta-glucosidase production in tobacco leaf allows to obtain enzyme quantities below the level of industrial attractiveness. Moreover, as indicated in WO'353, the enzyme quantities that can be purified from the leaf biomass are even smaller when expressed in terms of enzymatic activity, supporting the existence of technological constraints in the leaf expression system, particularly in connection with the use of constitutive promoters.

In WO'839, a tentative solution of the problems encountered with beta-glucosidase production in plants is presented. In particular, WO'839 describes the outcomes of tobacco transformation with an expression vector containing the gene encoding a mutated form of beta-glucosidase operatively linked with the 7S soy globulin promoter and a nucleotide sequence coding for the 7S soy globulin signal peptide. By means of immunological assay, the polypeptide of interest is actually detected in raw seed extracts.

In the WO'839 description, no mention is made to the phytotoxic effects on transformed tobacco plants or on their progenies caused by beta-glucosidase accumulation in the seed, therefore the system may appear to be effective in solving the problems related to the production in plants of lysosomal enzymes and, in particular, beta-glucosidase.

However, subsequent experiments carried out on the seed produced by transformed tobacco plants revealed that beta-glucosidase accumulation determines important rebounds on seed viability. In particular, it has been demonstrated that, already at enzyme concentrations close to 200 U/kg of seed (1 U=amount of enzyme releasing one micromole of 4-methylumbelliferyl-D-glucoside per minute at 37° C., pH 5.9), there are reproductive anomalies caused by a low viability and a stunted germination. Moreover, above roughly 500 U/kg of seed, seed viability is completely impaired.

Further experiments carried out by the Applicant have shown that seed viability of tobacco transformed with the same construct cited in WO'839 cannot be restored in any known way. An Applicant investigation performed by electron microscopy has revealed a disorganization and a destructuration of the cell membrane system in unviable seed, confirming the inability to obtain a vital progeny from those transgenic lines that may theoretically be exploited for the industrial production of the enzyme. It was subsequently verified by electron microscopy that the storage site of the mutated form of beta-glucosidase corresponds to the storage protein vacuoles internal to tobacco embryo parenchimatic cells.

In general, although in line of principle the plants needed for the production of industrial enzyme quantities could be obtained by in vitro propagation of elite plants, the use of this system would unavoidably complicate the production cycle due to the need for a large number of plants to be transplanted in the field in a relatively short time. In order to plan a 60 ton production of transgenic tobacco seed to satisfy Italy's human beta-glucosidase demand, an investment in at least 150 hectares of land and, above all, the in vitro production, greenhouse acclimatization and field transplantation of at least 9 million tobacco plants would be needed.

With regard to process management and economic issues, this is clearly a very different situation from that resulting form transgenic seed broadcasting or sowing in narrow rows; these latter operations, feasible only with viable seed, would also allow a fourfold-sixfold higher seed production per unit of area compared to standard tobacco crops in view of the much higher plant density. The achievement of a plant density similar to that obtainable with direct sowing is unthinkable by transplantation as its cost would be insufficiently compensated from a productive point of view; in fact, there is an inverse proportion between plant production and plant number per unit of area. As to micropropagation costs, each plant should be handled individually and this, combined with the very low seed quantity (a few grams) produced by each tobacco plant, would drastically decrease the benefits of exploiting plants as host systems for lysosomal enzyme production.

Hence, WO'839 leaves unresolved several essential issues:
lysosomal enzymes still exert unwanted phytotoxic effects on plants, in particular on their reproductive behaviour, forcing the adoption of a cumbersome and expensive procedure in the application of the proposed technology;
the productive lines cannot be maintained and multiplied through the natural process of sexual reproduction and cultivation of the resulting progenies;
it is therefore impossible to establish master seed banks and working seed banks as requested by the existing national and international legislation dealing with good manufacturing practice of active substances for therapeutic use;
rebuilding the route of production on each occasion would mean retesting every time the seed produced by each transformed plant (i.e. few grams) and bulking the selected seed but this is clearly unaccepted by all regulatory agencies such as EMA or FDA;
in vitro propagation as well as cryopreservation of the productive lines can result in mutations or in the instability of the genetic construct inserted into the plant genome.

In WO'839 the examples dealing with the construction of expression vectors for lysosomal enzyme production always report the use of storage protein promoters of dicot plants. The examples concerning the actual expression of lysosomal enzymes are limited to a mutated form of acid beta-glucosidase and the host plant used is always tobacco (Nicotiana tabacum L.).

All this considered, WO'839 neither provides the teachings for the production of lysosomal enzymes, and in particular beta-glucosidase, in plants nor does it give information on how to elude, minimize and overcome the problems and the consequent limitations connected with tissue expression and subcellular localization of such enzymes, and in particular of beta-glucosidase, in the seed.

A further problem involves the extraction and purification of lysosomal enzymes from the seed according to the teachings contained in WO'839. According to WO'839, seed should be homogenized in liquid nitrogen and the resulting crude extract partially purified by ultrafiltration and processed through HPLC. None of these methods is novel and at the same time none can be applied in industry: in fact, grinding a seed sample in a buffer with liquid nitrogen is not feasible if the buffer volume is even slightly larger than a few litres; similarly, ultrafiltration of a large volumes of crude extract cannot be performed because of membrane clogging. Finally, HPLC procedures are not industrially applied due to the fact that only tiny samples can be processed at each run. Hence, WO'839 does not contains the teachings for the industrial production of lysosomal enzymes in a purified form, which is the only suitable for therapeutic use. Actually, in WO'839 no real demonstration of enzyme purification from the seed is provided, not even at a laboratory scale. In conclusion, in spite of the premises, WO'839 does not constitute a possible solution to the production of beta-glucosidase in plants, even less of other lysosomal enzymes, and a person skilled in the art would certainly fail in applying the invention of WO'839, at least at an industrial scale.

Similarly to WO'839, patent application WO-A-00/04146 (WO'146), which describes the expression of human lactoferrin and of proteins with a generic enzymatic activity in the plant seed, does not provide any teaching about the production of functionally active enzymes, least of all of lysosomal enzymes. In WO'146, no evidence is provided to support the effectiveness of the enzyme production process and problems related to the stability, conformation and functionality of said enzymes are totally neglected.

### BRIEF SUMMARY OF THE INVENTION

A purpose of the present invention is to carry out a method for the production of recombinant human lysosomal enzymes in a cereal endosperm, particularly acid beta-glucosidase and acid alpha-glucosidase in rice endosperm. The newly devised method overcomes the difficulties proper of the known technological state, more specifically:
it allows an effective tissue expression and subcellular localization of lysosomal enzymes, and, in particular, of human acid beta-glucosidase and human acid alpha-glucosidase, inside the seed;
it allows the production of lysosomal enzymes suitable for therapeutic use;
it eliminates the risk of phytotoxic phenomena;
it does not affect seed viability;
it enables the creation of master- and working seed banks;
it enables the maintenance and multiplication of productive lines through sexual reproduction;
it facilitates both enzyme extraction and purification;
it is economically more convenient;
it greatly facilitates the management of the productive process;
it eliminates the risk of partial enzyme degradation;
it greatly reduces the level of bacterial and fungal contamination.

The Applicant has devised, tested and embodied the present invention to overcome the shortcomings of the state of the art and to obtain these and other purposes and advantages.

The present invention is set forth and characterized in the independent claims, while the relative dependent claims describe other characteristics of the invention or variants to the main inventive idea.

In accordance with the above-mentioned aims, a method for the production of recombinant human lysosomal enzymes, suitable for therapeutic use, in a cereal endosperm, comprises:

the construction of a plant expression vector for the transformation of such cereal, containing a nucleotide sequence harbouring the following elements:
i) the rice glutelin 4 promoter (GluB4pro) as an endosperm-specific promoter upstream the gene encoding said lysosomal enzyme;
ii) the leader known as LLTCK as a 5′ UTR region;
iii) the PSGluB4 sequence encoding a signal peptide used in rice to target the precursor of glutelin 4 inside the endoplasmic reticulum, said signal peptide being suitable to carry out a co-translational transfer of the newly synthesized lysosomal enzyme into the lumen of the endoplasmic reticulum of the endosperm cells and to determine the accumulation of said lysosomal enzyme in a specific cell compartment;
iv) a nucleotide sequence encoding the mature form of the human lysosomal enzyme;
v) a 3′ UTR of natural or artificial origin;
a step of cereal plant transformation using said vector, whereby the lysosomal enzyme is obtained and confined in an endosperm, which is not eventually absorbed by the embryo, and the presence of large quantities of the lysosomal enzyme in the endosperm does not negatively affect seed viability and germination speed;
a step of lysosomal enzyme accumulation inside the seed endosperm of said cereal plant.

The present invention allows the accumulation of a heterologous recombinant human lysosomal enzyme in storage tissues not belonging to the seed embryo. The present invention also favours the accumulation of a recombinant human lysosomal enzyme with a high phytotoxic/destructurating potential in storage tissues not belonging to the seed embryo and spontaneously undertaking an apoptotic process at the end of development.

Moreover, it is possible to accumulate potentially phytotoxic proteins in non-vital tissues which will be subject to an intense hydrolytic activity after seed imbibition and germination.

These potentially dangerous proteins can be accumulated within the protein storage vacuoles or protein bodies without being in contact with or crossing the cell membrane.

The present invention allows the production of exactly the intended amino acid sequences rather than non-authentic variants of the protein characterized by the presence of additional amino acids which are useless if not potentially harmful in terms of protein trafficking, stability, biological activity and therapeutic use. The synthesized protein is advantageously accumulated in the endosperm within protein storage vacuoles (PSVs) or protein bodies (PBs). Since protein extractability from PSVs or from PBs is rather similar, the localization of said protein in one or the other of the above-cited subcellular compartments is indifferent in terms of the validity of the present invention.

An embodiment of this invention implies the construction of an expression vector for cereal plant transformation which comprises a sequence harbouring the following elements:

i) the rice glutelin 4 promoter (GluB4pro) as an endosperm-specific promoter upstream the gene encoding said lysosomal enzyme;

ii) the leader known as LLTCK as a 5′ UTR region;

iii) the PSGluB4 sequence encoding a signal peptide used in rice to target the precursor of glutelin 4 inside the endoplasmic reticulum, said signal peptide being suitable to carry out a co-translational transfer of the newly synthesized lysosomal enzyme into the lumen of the endoplasmic reticulum of the endosperm cells and to determine the accumulation of said lysosomal enzyme in a specific cell compartment;

iv) a nucleotide sequence of natural or artificial origin encoding the mature form of the recombinant human lysosomal enzyme;

v) a 3′ UTR of natural or artificial origin;

and the use of such vector for cereal plant transformation.

Advantageously, the nucleotide sequence of the expression vector is as reported in SEQ ID No: 1.

According to an embodiment of this invention, the expression vector is introduced into bacterial strains, which are directly or indirectly used for plant transformation. Advantageously, the selected bacterial strain belongs to a group which comprises Escherichia coli, Agrobacterium tumefaciens and Agrobacterium rhizogenes.

According to a preferred embodiment, the bacterial strain is used for transformation of embryogenic calli of rice (Oryza sativa ssp. japonica, var. CR W3). According to an advantageous variant, the lysosomal enzyme is the human acid beta-glucosidase. In another variant, the lysosomal enzyme is the human acid alpha-glucosidase.

Actually, the present invention is equally effective in the synthesis, extraction and purification of significant amounts of human acid alpha-glucosidase precursor, which has a molecular mass, structure and function that is completely different from acid beta-glucosidase.

According to an embodiment, the present invention comprises a third step consisting in industrial seed manufacturing.

According to a solution, the industrial manufacturing is designed to dehull and whiten the harvested mature seed in order to remove the fibrous components, the germ and the aleuronic layer containing a number of protein contaminants.

According to a further embodiment, the present invention comprises a fourth step of purification of the recombinant human lysosomal enzyme. The purification step preferably consists in a hydrophobic interaction chromatography, an ion exchange chromatography and a gel filtration, in that order. Moreover, the purification step may include, alternatively or additionally, the application of chromatographic resins with a chemical composition and/or structure and/or function similar to those indicated in the examples hereinafter reported, the partial modification of the elution conditions, the duplication of a passage, e.g. by reloading an eluted fraction into a column.

With the present invention the recombinant human lysosomal enzyme is purified in amounts which are easily larger than 100 U/kg of seed, or even up to 500 U/kg of seed. In addition, the purified enzyme is extremely active, it does not present deletions, additions or amino acid substitutions, resulting in this respect perfectly equal to the human native counterpart. Moreover, the accumulation of the enzyme in the endosperm does not determine any alteration of seed viability or germination speed.

The molecular cassette used for enzyme production is normally inherited by the progenies and, as any Mendelian factor, can be brought to homozygosis or transferred by crossing to other transformed lines, favouring in both cases an increase in enzyme production.

The method related to the present invention, contrary to known technologies, is clearly innovative and advantageous because it allows to obtain transgenic lines, for example of rice, which are able to produce industrially relevant amounts of recombinant human lysosomal enzymes suitable for therapeutic use, in particular human acid alpha or beta-glucosidase, showing no alteration to the normal phenotype (both at a macroscopic and microscopic level) and in particular no reproductive anomaly or alteration in seed viability and germination speed, also with enzyme concentrations higher than 500 U/kg of seed. The method also allows to extract and purify the enzyme in a completely active form, maintaining the amino acid sequence unchanged as regards the human native counterpart.

Falling within the present invention is a nucleotide sequence suitable for the expression of recombinant human lysosomal enzymes, suitable for therapeutic use, in a cereal endosperm; said nucleotide sequence comprises the following elements:

i) the rice glutelin 4 promoter (GluB4pro) as an endosperm-specific promoter upstream the gene encoding said lysosomal enzyme;

ii) the leader known as LLTCK as a 5′ UTR region;

iii) the PSGluB4 sequence encoding a signal peptide used in rice to target the precursor of glutelin 4 inside the endoplasmic reticulum, said signal peptide being suitable to carry out a co-translational transfer of the newly synthesized lysosomal enzyme into the lumen of the endoplasmic reticulum of the endosperm cells and to determine the accumulation of said lysosomal enzyme in a specific cell compartment;

iv) a nucleotide sequence of natural or artificial origin encoding the mature form of the human lysosomal enzyme;

v) a 3′ UTR of natural or artificial origin.

According to the present invention, the sequence of the rice glutelin 4 promoter (GluB4pro) is indicated in SEQ ID No: 2.

According to an advantageous embodiment, the 5′ UTR ii) is the leader known as LLTCK, described in patent application PCT/EP2007/064590 and reported in SEQ ID No: 3.

According to the present invention, the nucleotide sequence of PSGluB4 encoding the signal peptide used by rice to target the glutelin 4 precursor into the endoplasmic reticulum is indicated in SEQ ID No: 4.

According to an embodiment of the present invention, the nucleotide sequence of the element iv) is the GCase sequence, encoding the mature form of the human acid beta-glucosidase, as indicated in SEQ ID No: 5.

According to an advantageous embodiment of the invention, the 3′ UTR of element v) is the NOS terminator, the sequence of which is indicated in SEQ ID No: 6. Alternatively, the terminator of GluB4 gene can be used.

According to an embodiment of the present invention, the whole nucleotide sequence of the expression cassette is the same as that reported in SEQ ID No: 1.

Falling within the present invention are the nucleotide sequences complementary to those above-mentioned.

Falling within the present invention are the sequences derived from mutagenic processes, such as deletions, insertions, transitions, transversions of one or more nucleotides of the above-mentioned sequences or of their complementary sequences provided that they maintain their function.

Falling within the present invention are the combinations of the above-mentioned sequences encoding the mature form of human acid beta-glucosidase with promoter elements and/or sequences for protein targeting to the endoplasmic reticulum and/or untranslated regions in 5′ and 3′ different from those reported in the sequence as indicated in SEQ ID No: 1, suitable to obtain the synthesis and accumulation of the enzyme specifically in the seed endosperm, or with nucleotide sequences complementary to said sequences.

Falling within the present invention are the combinations of the elements i), ii), iii), iv) and v) as described above with mature enzyme encoding sequences different from those reported in SEQ ID No: 1 for the presence of mutations or polymorphisms internal to the human species or combinations made with their complementary sequences.

Moreover, falling within the present invention are also the combinations of the elements i), ii), iii), iv) and v) as mentioned above with sequences encoding mature forms or precursors of other lysosomal enzymes, or combinations made with their complementary sequences.

Furthermore, falling within the present invention are also the above-cited combinations, in which the enzyme is the human acid alpha-glucosidase.

Falling within the present invention is also a sequence as mentioned above, in which the transformed plants are cereals.

Falling within the present invention is a molecular vector for the expression of a human lysosomal enzyme in a plant endosperm, harbouring said nucleotide sequence. Typically, the molecular expression vector is a plasmid.

According to an advantageous solution, the lysosomal enzyme is the human acid beta-glucosidase.

Alternatively, the lysosomal enzyme is the human acid alpha-glucosidase.

Falling within the present invention is also the use of the above-cited expression vector for plant transformation with the aim to produce a human lysosomal enzyme.

Falling within the present invention is also a bacterial strain containing the expression vectors as described above. Advantageously, that bacterial strain can be chosen from a group comprising Escherichia coli, Agrobacterium tumefaciens and Agrobacterium rhizogenes.

Falling within the present invention are the plant cells transformed with expression vectors as those cited above.

According to a solution of the present invention, those cells are cereal cells, preferably belonging to cultivated rice (Oryza sativa L.). There is a preference for rice varieties unsuitable for use as food. Hence, falling within the present invention is the use of waxy rice, industrially exploitable for the extraction and production of starch and its by-products.

Alternatively, cells may belong to a member of the Graminaceae family (Poaceae), e.g. maize (Zea mays L.), barley (Hordeum vulgare L.) and wheat (Triticum spp.).

Falling within the present invention is also the seed of a plant transformed for the expression of a human lysosomal enzyme, which contains an expression vector as described above.

According to a solution of the invention, the seed of the transformed plant belongs to a cereal species, preferably the transformed plant belongs to the rice species Oryza sativa L.

The field of protection related to the present invention also comprises a transformed plant for the expression of a human lysosomal enzyme, obtained with the use of an expression vector as mentioned above. Advantageously, such plant is a cereal, preferably belonging to the rice species Oryza sativa L.

Falling within the present invention are also the progenies obtained by self-fertilization or crossing, or transformed lines selected from the above-mentioned transformed plant.

The present invention also refers to a seed as described above for therapeutic treatment. Moreover, the invention also refers to the use of the aforementioned seed for the production of an ERT drug. In particular, it refers to enzyme replacement therapy for the following diseases: Gaucher disease, Glycogenosis type II, Fabry disease, Niemann-Pick B disease, Mucopolysaccharidoses I, II, IV.

The invention also refers to a seed as cited above to be used in enzyme replacement therapy. In particular, the invention refers to a seed as mentioned above to be used in the enzyme replacement therapy of the following diseases: Gaucher disease, Glycogenosis type II, Fabry disease, Niemann-Pick B disease, Mucopolysaccharidoses I, II, IV.

### BRIEF DESCRIPTION OF THE SEVERAL VIEWS OF THE DRAWINGS

These and other characteristics of the present invention will become apparent from the following description of a preferential form of embodiment, given as a non-restrictive example with reference to the attached drawings wherein:

FIG. 1 is a scheme of the final expression vector pSV2006[GluB4pro/LLTCK/PSGluB4/GCase/NOSter] used for the endosperm-specific production of the human enzyme acid beta-glucosidase;

FIG. 2A shows an experimental scheme of the method for the synthesis by recursive-PCR of the LLTCK leader downstream the GluB4 promoter;

FIG. 2B shows the results of electrophoretic analyses of duplex-PCR products obtained from genomic DNA extracted from putatively transformed plants with primer couples annealing to the GCase and HPT II genes. Lane 1: 1 Kb ladder (NEB); lane 2: negative control (NC), i.e. genomic DNA extracted from a non-transformed plant; lane 3: positive control (PC), i.e. pSV2006[GluB4pro/LLTCK/PSGluB4/GCase/NOSter] vector; lanes 4-16: tested plants;

FIGS. 3A and 3B show the results of SDS-PAGE (A) and Western blot (B) analyses carried out on protein extracts obtained in the course of extraction trials from seed of GCase transformants. In A and B, lanes 1-5 are loaded with serial consecutive extractions of the whitened rice sample, lanes 6 and 7 with two consecutive extractions of the whitening waste. Positive control (PC): in Western blotting, it corresponds to 100 ng of purified imiglucerase. It is evident that most of the recombinant human acid beta-glucosidase contained in whitened rice can be recovered with three serial extractions;

FIG. 4A shows the results of Western blot analyses carried out on protein extracts obtained from seed of GCase transformants. Lane 1: marker Precision Plus Protein standard (BioRad); lane 2: positive control (PC, 100 ng imiglucerase); lane 3: negative control (NC, protein extract from non-transformed rice, var. CR W3); lanes 4-10: seed protein extracts of different primary transformants;

FIG. 4B shows the three glycoforms of human acid beta-glucosidase detected with Western blot analysis after a 2-dimensional electrophoresis of a seed protein extract from a GCase transformed plant;

FIGS. 5A and 5B report an image of immunolocalization obtained by transmission electron microscopy (magnification 12500×) on a seed section of a non-transformed rice (A) and a GCase transformant (B). It is evident that the accumulation of recombinant human acid beta-glucosidase involves only the protein storage vacuoles (PSVs);

FIGS. 6A and 6B shows an example of HIC (A) and IEC (B) chromatograms where the elution peaks containing the recombinant human acid beta-glucosidase are indicated;

FIG. 7 reports in a graph the fluorescence recorded in 4-MUG assays carried out with different chromatographic aliquots relative to NC (non-transformed plant) and a GCase transformant. The results of the associated Western blot analyses are also reported. EX: raw extract; R: flow through; E: elution aliquots; PC: positive control (imiglucerase). It is evident that the true GCase activity (E2-E3) can be separated from the endogenous GCase-like one (E6) with IEC;

FIGS. 8A and 8B show the results of a SDS-PAGE analysis (A) carried out on recombinant human acid beta-glucosidase after the final purification step with gel filtration and the corresponding Western blot signal (B);

FIG. 9 reports the mass spectrum obtained by MALDI-TOF analysis on a GCase sample purified by HIC and IEC;

FIG. 10 shows a schematic representation of GAA gene assembling in pUC18 from the initial artificially-synthesized fragments;

FIG. 11 shows a schematic representation of the strategy adopted to achieve the final expression vector pSV2006[GluB4pro/LLTCK/GAA/NOSter];

FIG. 12 shows the results of a Western blot analysis carried out on total protein extracts obtained from different GAA transformants. Lane 1: M, marker Precision Plus Protein standard (BioRad); lane 2: NC (seed protein extract from a non-transformed plant); lane 3: PC (100 ng of Myozyme); lanes 4-10: seed protein extracts obtained from different primary transformants; and

FIG. 13 shows the results of an immunogold labelling of mature seed endosperm carried out with an anti-GAA antibody. It is evident that GAA is specifically detected in the protein storage vacuoles (PSVs) and not in the protein bodies (PBs). No signal was ever detected in the negative control (seed produced by an untransformed plant) (data not shown). Magnification: 16000×.

### DETAILED DESCRIPTION OF THE INVENTION

The present invention refers, in particular, to a method for the production of human acid beta-glucosidase in the seed endosperm of cultivated rice (Oryza sativa L.); the method comprises:
a first step of plant transformation whereby the recombinant human lysosomal enzyme is obtained and confined in an endosperm, which is not eventually absorbed by the embryo, and the presence of large quantities of the lysosomal enzyme in the endosperm does not negatively affect seed viability and germination speed;
the use, in the first step of plant transformation, of an endosperm-specific promoter upstream the gene encoding said lysosomal enzyme, and of a signal peptide for a co-translational transfer of the newly synthesized lysosomal enzyme into the lumen of the endoplasmic reticulum of the endosperm cells for its tissue-specific accumulation;
a second step of protein accumulation inside the seed endosperm of a plant.

In the plant transformation method, the use of an expression vector containing the following elements is envisaged:

i) an endosperm-specific promoter of natural or artificial origin;

ii) a 5′ UTR of natural or artificial origin;

iii) a nucleotide sequence of natural or artificial origin encoding a signal peptide suitable to target the recombinant lysosomal enzyme into the lumen of the endoplasmic reticulum of the endosperm cells and to determine the accumulation of said lysosomal enzyme in a specific tissue;

iv) a nucleotide sequence of natural or artificial origin encoding the mature form of the human lysosomal enzyme;

v) a 3′ UTR of natural or artificial origin.

The nucleotide sequence contained in the expression vector is, for example, that indicated in SEQ ID No: 1.

Among the possible known endosperm-specific promoters of Graminaceae plants and in particular of rice, the present invention advantageously exploits the promoter of the GluB4 gene (the sequence of which is reported in SEQ ID No: 2), because the GluB4-encoded lysosomal enzyme presents a more uniform distribution inside the seed endosperm. Moreover, the GluB4 promoter has a higher transcriptional activity compared to promoters of genes encoding other storage proteins within rice endosperm, like globulins, prolamins, or glutelins other than GluB4.

The GluB4 promoter was isolated by PCR from the waxy rice variety CR W3 (selected by Ente Nazionale Risi, Milan) together with its leader region. Since the native leader is rather short and scarce in repeated CAA and CT elements, which have a positive influence on gene expression, it was eventually substituted with the 5′ UTR known as LLTCK (De Amicis et al. 2007, Transgenic Res 16: 731-738) and reported in the international patent application PCT/EP2007/064590 and indicated in SEQ ID No: 3.

The sequence GluB4pro/LLTCK was ligated with PSGluB4/GCase, where:
PSGluB4 is the sequence (as indicated in SEQ ID No: 4) encoding the signal peptide used by rice glutelin 4 precursor to enter the endoplasmic reticulum;
GCase is the sequence (as indicated in SEQ ID No: 5) encoding the mature form of human acid beta-glucosidase; the mature form consists in the precursor protein deprived of the native signal peptide.

In order to avoid the addition of foreign amino acids at the N-terminus of the mature protein, which could result from the introduction of endonuclease restriction sites eventually used for cloning or sequence connection, the DNA region corresponding to the PSGluB4 sequence and the initial part of the mature GCase coding sequence (until the naturally-occurring Hind III restriction site) was artificially synthesized.

The sequence of PSGluB4 resulting from such synthesis does not match with the natural rice sequence, although it is fully synonymous, due to changes that have been deliberately introduced to favour a better recognition of the translation start codon in association with LLTCK and to avoid the occurrence of rare codons or unfavourable intercodon contexts. On the contrary, the GCase initial part was maintained unaltered with respect to the human native sequence, so the whole GCase sequence corresponds exactly to the GenBank accession No M16328, in the interval between nucleotide positions 553 and 2046. After connection of the synthetic sequence with that encoding the remaining part of the enzyme through the Hind III restriction site, the whole complex was ligated at the 3′ terminus of GluB4pro/LLTCK, previously cloned in the pSV2006 binary vector. pSV2006 was developed by the Applicant from pCAMBIA 1300 plasmid (www.cambia.org); the polyadenilation signal used for the human acid beta-glucosidase construct was NOS ter, i.e. the terminator of Agrobacterium tumefaciens nopaline synthase gene. The NOS terminator sequence is reported in SEQ ID No: 6.

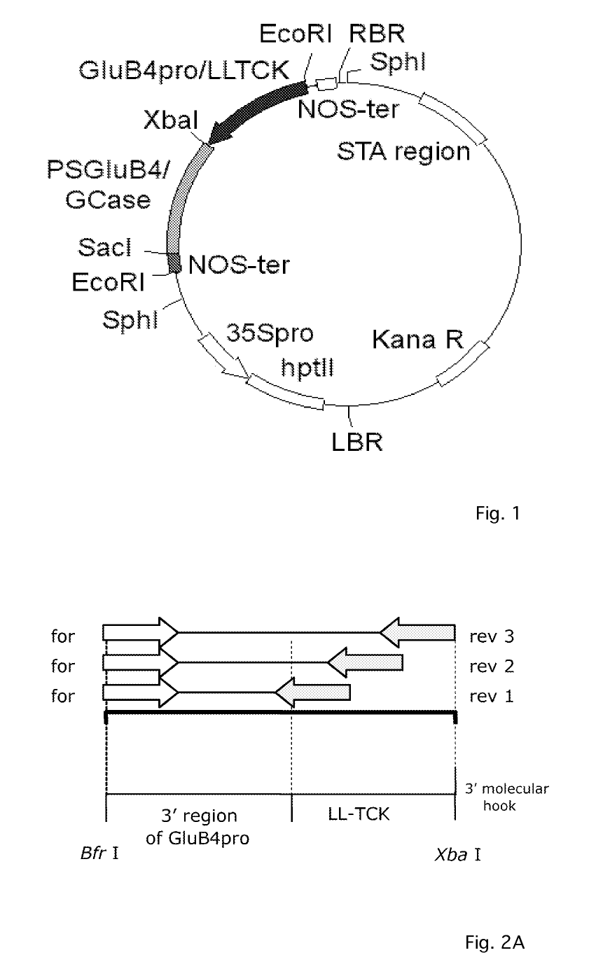

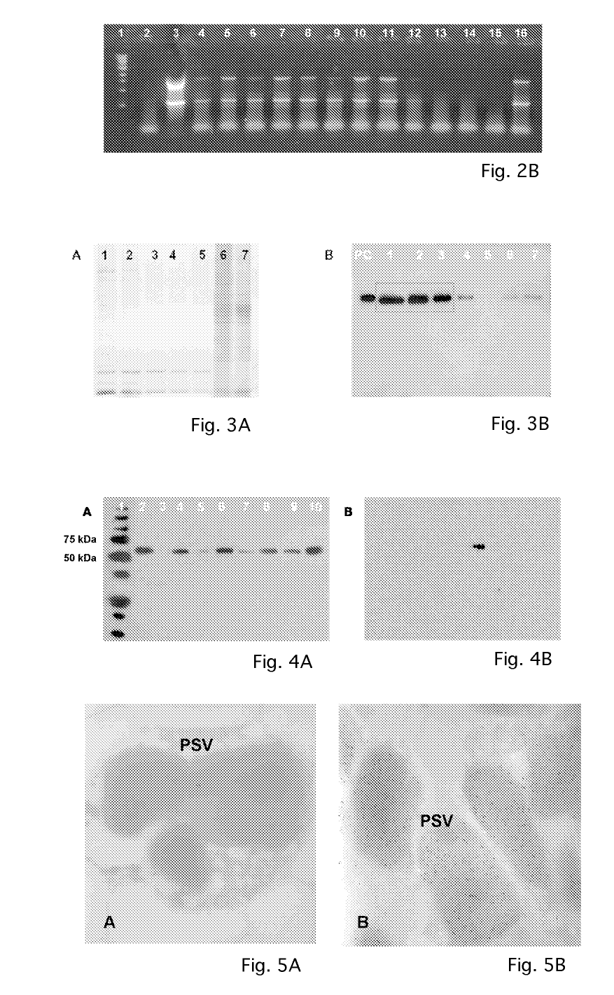

After checking all the sequences used in the construction of the final vector pSV2006[GluB4pro/LLTCK/PSGluB4/GCase/NOSter] (FIG. 1), this vector was introduced into the EHA 105 strain of Agrobacterium tumefaciens by electroporation. Then, the engineered strain was used for the transformation of rice embryogenic calli (Oryza sativa ssp. japonica, var. CR W3). The whole procedure of plant transformation and regeneration on selective medium was regularly completed. No differences were observed between transformed and control plants grown in climatic chambers under the same conditions of light, temperature and humidity. The female and male organ fertility and the percentage of flower abortion in transgenic plants were found comparable with those observed in non-transformed plants of the CR W3 variety. All primary transformants produced seed with a mean viability higher than 95%, irrespectively of the level of human acid beta-glucosidase expression. Moreover, the germination speed matched the maximum values of the species (within 4-6 days, almost all of the viable seed developed primary roots and the coleoptile). Similarly to primary transformants, also their progenies grew normally and produced seed containing recombinant human acid beta-glucosidase. The presence of the enzyme encoding gene was verified by PCR analyses in all putatively transformed plants (FIG. 2B) and in over 150 randomly-sampled progenies of the best primary transformants obtained by selfing. In these analyses, negative and positive controls, corresponding respectively to total DNA of non-transformed CR W3 plants and to miniprep extractions of the expression vector, were used. Furthermore, the amplifiability of each tested DNA extract was also demonstrated, making use of a specific primer couple designed on a rice chloroplastic DNA region. On the whole, PCR analyses demonstrated that the rice genome is transformed with the sequence encoding the human acid beta-glucosidase enzyme and the transgene is transmitted to progenies. The hereditary transmission was demonstrated not only in selfed progenies but also in those derived by crossing transformed plants with negative controls or with other transformed plants. In order to verify the production of the human acid beta-glucosidase messenger RNA, immature rice seeds (10-15 days after flowering) were harvested and used for total RNA extraction. On the latter, the following analyses were performed: a. absence of genomic DNA contamination by PCR; b. amplification of the human acid beta-glucosidase messenger RNA by RT-PCR; c. amplification of the glutelin 4 messenger RNA by RT-PCR. In all the cases, the GCase gene appeared regularly expressed and showed the same expression pattern of the gene encoding the glutelin 4 storage protein. As expected, when total RNA from negative control was used, only the amplification of glutelin 4 gene was obtained.

Immature seed was also used to immunolocalize the recombinant protein by transmission electron microscopy. This work demonstrated that the human acid beta-glucosidase is accumulated exclusively in the protein storage vacuoles of the endosperm cells. When the same analysis was repeated on the CR W3 control seed, no signal was obtained; this demonstrated the great effectiveness of the analysis and the absolute specificity of the anti-GCase antibody we used. The availability of a specific antibody together with the possibility to measure beta-glucosidase activity through a reliable and sensitive fluorimetric assay were exploited to select the best transgenic lines and to develop a purification procedure of the recombinant lysosomal enzyme. Concerning the extraction and purification processes, protocols were developed for preliminary seed manufacturing, crude protein extraction and recombinant acid beta-glucosidase isolation and purification. The purification protocol consists in three serial steps: a hydrophobic interaction chromatography (HIC), a cation exchange chromatography (IEC) and gel filtration (GF). Seed dehulling and whitening were absolutely useful for removing the large part of protein contaminants with a minimal GCase loss; losses were also very low during the extraction steps. The removal of the endogenous GCase-like enzyme, responsible for a GCase-like activity, was obtained through a discontinuous elution process applied at the end of cation exchange chromatography. This chromatography allowed a further decrease of protein contaminants assigning to the gel filtration step the role of sample polishing.

In SDS-PAGE, the purified protein showed essentially the same mobility of imiglucerase (Cerezyme, Genzyme Corp.) and an apparent molecular weight of about 60 kDa. In Western blotting, the purified protein was strongly detected by an anti-imiglucerase antibody raised in rabbit. The purified protein was found to be enzymatically active; in particular, it efficiently hydrolyzed the fluorogenic substrate 4-methylumbelliferyl beta-D-glucoside, showing the same reaction kinetic of imiglucerase. In 2-D electrophoresis, the single band repeatedly detected in Western blot analyses carried out after standard SDS-PAGE split in at least three protein glycoforms. Further analyses demonstrated the integrity of recombinant human acid beta-glucosidase produced in rice endosperm and the identity of its amino acid sequence with the human native counterpart. N-terminus microsequencing demonstrated that the initial nonapeptide corresponds to ARPCIPKSF which is also the N-terminus of human native acid beta-glucosidase. Peptide mass fingerprinting performed in MALDI-TOF showed that also the C-terminus of the protein is fully conserved and that, similarly to what was observed in the native enzyme, no glycan chains are found in the fifth N-glycosylation site. Differently, the first, second, third and fourth N-glycosylation site of the protein appeared to be occupied. The presence of N-glycans in the first site is essential for the enzymatic activity of the protein.

### EXAMPLES

### Example 1

### Construction of the Molecular Cassette for GCase Expression

The following section describes a method for the endosperm-specific expression of human acid beta-glucosidase in rice. Similar methods can be used to carry out variants of the construct, characterized by the presence of other endosperm-specific promoters and/or sequences for protein targeting into the endoplasmic reticulum.

Isolation of the Glutelin 4 Promoter (GluB4pro)

In order to isolate the Glutelin 4 promoter of Oryza sativa (GenBank acc. No AY427571), a PCR on genomic DNA of CR W3 variety was performed. In such PCR, the following primers were used:
Primer GluB4pro for: as indicated in sequence SEQ ID No: 7.
Primer GluB4pro rev: as indicated in sequence SEQ ID No: 8.

In order to favour subsequent cloning, the GluB4pro for primer was designed to insert the Sph I and Eco RI restriction sites at the 5′ end of the amplicon; similarly, the GluB4pro rev primer was designed to introduce a Xba I site at the 3′ end of the PCR product.

Cycle: 95° C. for 2′; 40×(95° C. for 45″; 63° C. for 40″; 72° C. for 2′); 72° C. for 5′.

The amplified product was cloned into pGEM-T (Promega) and fully sequenced.

Substitution of native leader with LLTCK artificial leader in GluB4 promoter

In order to substitute the native leader of rice Glutelin 4 promoter (GluB4pro) with the synthetic leader LLTCK (De Amicis et al. 2007, Transgenic Res 16: 731-738), three serial PCR were performed with suitable primers (one forward primer and three reverse primers) according to the scheme of FIG. 2A.

In the first PCR, the plasmid pGEM-T[GluB4pro] was used as template; in the following two, the template was the product of the previous reaction. The forward primer 1 starts with a Bfr I restriction site and anneals close to the 3′ end of the GluB4pro sequence. The reverse primer 1 anneals with its 3′ end to the GluB4pro region immediately upstream the leader region. The part which does not anneal contributes to the synthesis of the initial LLTCK tract. The reverse primer 2 anneals to the latter fragment and determines the synthesis of the second part of the LLTCK leader sequence. Finally, the reverse primer 3 introduces the terminal portion of the LLTCK sequence as well as a Xba I site at the 3′ edge. PCR reactions were carried out using the Accu Taq (Sigma) DNA polymerase and the following temperature cycling: 98° C. for 2′; 15 (I and II PCR) or 25(III PCR)×(94° C. for 30″; 65° C. for 30″; 68° C. for 1′); 68° C. for 10′. The final PCR product was cloned into pGEM-T and verified by enzymatic digestion and sequencing. In order to replace the GluB4pro native leader sequence with the artificial LLTCK leader, the Bfr I and Xba I restriction sites were used. Vector and insert were ligated with the T4 DNA ligase and the resulting vector pGEM-T[GluB4pro/LLTCK] was verified by PCR analyses and enzymatic digestion.

Substitution of the Native Signal Peptide with the SPGluB4

To increase GCase expression in rice, the nucleotide sequence encoding the signal peptide of glutelin 4 (SPGluB4) was optimized on the basis of rice codon usage and put in front of the sequence encoding the mature form of human acid beta-glucosidase (GCase). In order to prevent the addition of foreign amino acids at the N-terminus of the mature enzyme, the addition of spurious endonuclease restriction sites at the edges to be connected was avoided. To solve the problem, an artificial fragment including a Xba I site at the 5′ end, the SPGluB4 sequence and the GCase initial region till the naturally-occurring Hind III site was produced and cloned into pUC57 (Fermentas). After a check of the sequence, it was cloned in place of the fragment encoding the native signal peptide inside pGEM-T[GCase], i.e. the plasmid containing the entire sequence encoding the human acid beta-glucosidase as reported in GenBank No M16328. Both pUC57[SPGluB4] and pGEM-T[GCase] were digested with Xba I and Hind III in order to generate the insert and the vector backbone, respectively. These parts were jointed together with T4 DNA ligase to produce pGEM-T[SPGluB4/GCase] which was checked by enzymatic digestion.

Assembly of the Molecular Cassette for Human Acid Beta-Glucosidase Expression in Rice

The regions corresponding to GluB4pro/LLTCK and SPGluB4/GCase were subcloned in two steps into pUC18[NOSter]; this plasmid was derived from pUC18 (Pharmacia) by inclusion of the NOS polyadenylation sequence of Agrobacterium tumefaciens. For this purpose, the restriction sites introduced at the 5′- and 3′ end of each region were exploited, namely Sph I and Xba I for GluB4pro/LLTCK, Xba I and Sac I for SPGluB4/GCase.

Production of the pSV2006[GluB4pro/LLTCK/SPGluB4/GCase/NOSter] Vector

To obtain the final expression vector, pSV2006 (a pCAMBIA 1300 derivative) was used. Through Eco RI digestion, the original expression cassette of pSV2006 and the molecular construct contained in pUC18[GluB4pro/LLTCK/SPGluB4/GCase/NOSter] were removed. The pSV2006 backbone and the insert of interest were ligated each other to obtain the final expression vector (FIG. 1), which was subject of specific analyses before its transfer into Agrobacterium tumefaciens, strain EHA 105 by electroporation. The engineered Agrobacterium tumefaciens strain was used for transformation of Oryza sativa ssp. japonica, var. CR W3.

### Example 2

### Rice Transformation Via Agrobacterium tumefaciens

Rice transformation was carried out according to the Hiei's protocol (Hiei et al., 1994), modified by C. Huge (Rice Research Group, Institute of Plant Science, Leiden University) and E. Guiderdoni (Biotrop program, Cirad, Montpellier, France). The main phases of the procedure are briefly reported below:

Development of Embryogenic Calli

Rice transformation was performed using scutellum-derived embryogenic calli. In order to induce callus proliferation from the scutellum tissue, rice seed was dehulled, disinfected to eliminate potential pathogens and saprophyte contaminants, washed several times with sterile distilled water, dried on sterile blotting paper and transferred to Petri dishes containing the callus induction medium (CIM). Dishes were incubated at 28° C. for 7 days in the dark; after that period, scutelli were excised from the seedling and cultivated on CIM for 14 days at 28° C. in the dark. At the end of the induction period, callus masses were selected on the basis of the presence of tiny white calli. These last were transferred to fresh CIM and cultivated for 10 days to develop embryogenic callus suitable for transformation.

Co-Cultivation of Calli with Agrobacterium tumefaciens

In order to obtain a sufficient amount of Agrobacterium tumefaciens for transformation, the strain harbouring the expression vector was incubated for 3 days at 30° C. on LB agar. The layer of agrobacterium cells was collected and resuspended in the liquid co-cultivation medium (CCML) until an O.D.₆₀₀ of 1.00 was reached (approx. 3-5·10⁹ cells/mL). The best calli, i.e. those compact, white-coloured and 2 mm in diameter, were dipped into the bacterial suspension. After blotting onto sterile Whatman paper, calli were transferred onto co-cultivation medium (CCMS) at a density of 20 per high-edge Petri dish (Sarstedt) and incubated for 3 days at 25° C. in the dark.

Selection of Hygromycin-Resistant Calli

At the end of the co-cultivation period, calli were transferred onto selection medium I (SMI) and incubated at 28° C. for 2 weeks in the dark. The calli were eventually transferred onto selection medium II (SMII) and incubated for another week at the same conditions.

Plant Regeneration from Transformed Calli

The regeneration of transformed plants was reached through an appropriate hormonal stimulation. Embryogenic hygromycin-resistant calli were selected, transferred onto the pre-regeneration medium (PRM) and incubated inside high-edge Petri dishes at 28° C. for 1 week. Calli were then transferred onto regeneration medium (RM) in the number of 8-10 per Petri dish. Plant regeneration occurred at 28° C. for 3-4 weeks in the light. When plants were sufficiently developed to be separated from the callus (≧3 cm in height), they were transferred in culture tubes containing 25 mL of rooting medium (ROT). Tubes were maintained for about 3 weeks at 28° C. in the light. At the end of the regeneration process, plants were potted in peat and grown to maturity in a confined phytotrone at 24° C., 85% relative humidity, under metallic halogen lamps Osram Powerstar® HQI®-BT 400 W/D (photoperiod 16 h light/8 h dark).

### Example 3

### Total Protein Extraction from Rice Seeds Transformed with GCase Construct

Transgenic rice seeds were firstly dehulled and whitened with Satake TO-92 (Satake Corporation, Japan). Whitened rice seeds were then milled and the resulting flour was homogenized in the extraction buffer (50 mM sodium acetate, 350 mM NaCl, pH=5.5), using a ratio between buffer volume (mL) and flour weight (g) equal to 10:1.5. After incubation at 4° C. for 1 hour, samples were centrifuged at 14000×g for 45 minutes. After supernatants recovery, the remaining pellets were used for two further extractions with the same procedure. Protein extracts obtained from the whitened seed and whitening waste were both analysed in SDS-PAGE (FIGS. 3A and 3B) and Western blotting. These analyses demonstrated that most of the recombinant human acid beta-glucosidase is contained in whitened seed and that it can be efficiently recovered with three consecutive extractions.

### Example 4

### Western Blot Analysis and 2-D Electrophoresis on Total Protein Extracts of GCase Transformed Seed

Total protein extracts were separated in SDS-PAGE (Laemli, 1970) using a Mini Protean II apparatus (BioRad) and a 0.75-mm thick 10% polyacrylamide gel. Before loading, samples were denaturated at 100° C. for 5 minutes, without beta-mercaptoethanol. After SDS-PAGE, proteins were transferred on polyvinylidene difluoride membrane (PVDF, Immobilon-P^{SQ} by Millipore) with a Trans-Blot SD apparatus (BioRad) at 15V for 30 minutes. Sample were then hybridised with a polyclonal anti-GCase antibody produced by immunizing two rabbits with commercial imiglucerase. The following hybridization conditions were applied: incubation for 1 hour at room temperature using a dilution equal to 1:1000 in blocking solution (7.5% p/v Oxoid skim milk in PBS). After washes in PBS Tween 0.1% v/v, an anti-rabbit HRP-conjugated secondary antibody (Sigma, dilution 1:10000) was incubated for 1 hour at room temperature. Then chemiluminescence was developed with ECL Plus™ (GE Healthcare). To determine the molecular weight of the positive protein bands, the Precision Plus Protein standard (BioRad) was used together with HRP-conjugated Precision Strepactin antibody (BioRad)(FIG. 4A).

Two samples containing about 200 μg of seed total protein were analyzed by isoelectrofocusing and SDS-PAGE. The first analysis was performed with PROTEAN IEF focusing system (BioRad) and ReadyStrip IPG of 7 cm, with a non-linear pH range of 3-10 (BioRad). Protein extracts were precipitated with 2D Clean-Up kit (GE Healthcare) and resuspended with 130 μL of DeStreak Rehydratation solution (GE Healthcare) and 0.6% Byolites ampholytes 3-10 (BioRad). The following running conditions were applied:
step 1: 250 V for 15 minutes
step 2: 4000 V for 2 hours
step 3: 20000 V-hour for approx. 24 hours.

At the end of the run, strips were washed with two different equilibration buffers: equilibration buffer I (2% DTT, 2% SDS, 50 mM Tris-HCl, 6 M urea, 30% glycerol and 0.002% bromophenol blue, pH=8.8) for 15 minutes and equilibration buffer II (2.5% iodoacetamide, 2% SDS, 50 mM Tris-HCl, 6 M urea, 30% glycerol and 0.002% bromophenol blue, pH=8.8) for 20 minutes. Samples were run in the second dimension in two separate gels according to a standard SDS-PAGE protocol. One electrophoretic gel was stained with Colloidal Coomassie Blue (0.08% Coomassie Blue R-250, 1.6% orto-phosphoric acid, 8% ammonium sulphate, 20% methanol), while the other was analysed by Western blotting (FIG. 4B). The whole procedure was performed in parallel also for protein samples obtained from non-transformed CR W3 seeds.

### Example 5

### Determination of the GCase Storage Site by Immunolocalization

Transformed seeds in the late milky phase were harvested, dehulled, cut into fragments of 1 mm and fixed in 0.2% glutaraldehyde for 1 hour at room temperature. After a wash in 0.15 M phosphate buffer, a dehydratation with a gradient of absolute ethanol (from 25 to 100%) was performed. Dehydrated samples were embedded in LR White Resin (London Resin Co.) and finally polymerized at 60° C. for 24 hours. Sections (2-3 μm thick) were cut with a LKB Nova microtome (Reichter), placed on nickel mesh grids (Electron Microscopy Sciences), incubated for 15 minutes with a goat normal serum solution (Aurion), diluted 1:30 in buffer C (0.05 M Tris-HCl, pH 7.6, 0.2% BSA) and eventually hybridized for 1 hour at room temperature with the primary anti-GCase antibody diluted 1:500 in buffer C. After several washes in buffer B (0.5 M Tris-HCl, pH 7.6, 0.9% NaCl) with 0.1% Tween 20 w/v (6×5 minutes), sections were incubated for 1 hour at room temperature with the secondary antibody conjugated with colloidal gold (15 nm, Aurion) diluted 1:40 in buffer E (0.02 M Tris-HCl, pH 8.2 containing 0.9% NaCl and 1% BSA). At the end of hybridization, sections were washed and stained with uranyl acetate and lead citrate (Reynolds, 1963) and finally observed with Philips CM 10 transmission electron microscope (TEM).

The results obtained showed the presence of GCase only in protein storage vacuoles (PSVs) of seed endosperm; the polyclonal anti-GCase antibody allowed to identify GCase with a strong and clear signal, in the absence of significant background. The same analyses carried out on non-transformed rice grains confirmed the high specificity of the anti-GCase antibody by the lack of any evidence for matrix-associated cross-reacting sites (FIGS. 5A and 5B).

### Example 6

### Recombinant Human Acid Beta-Glucosidase Purification from Rice Seed

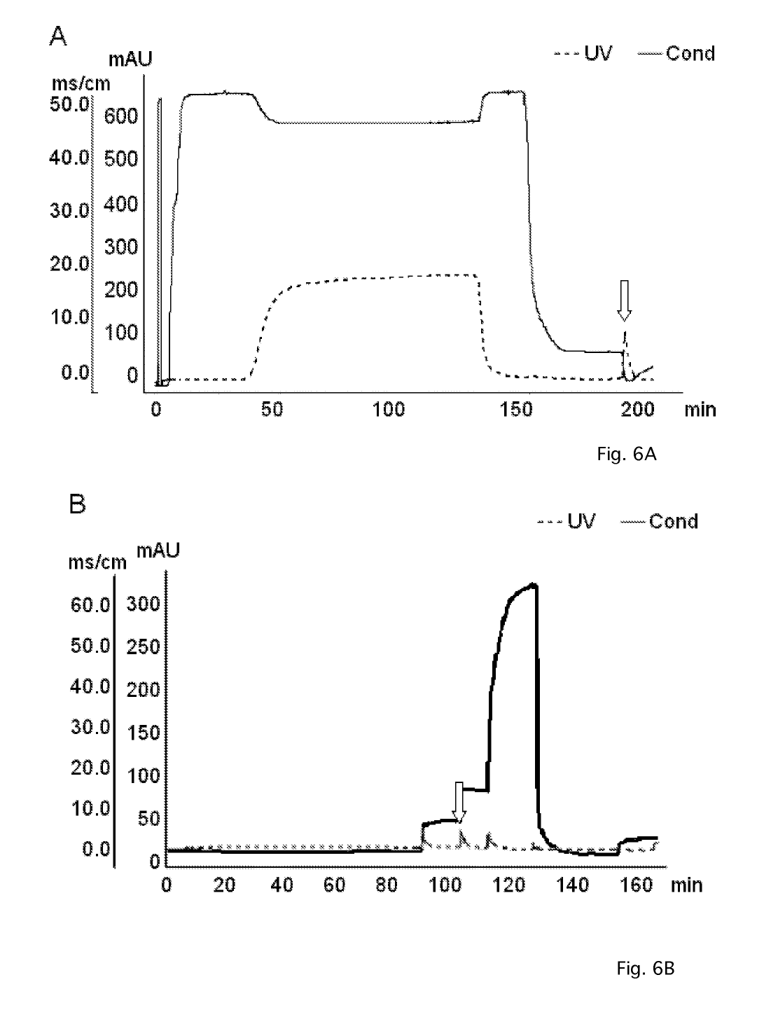

For the purpose of purification, an industrially-scalable protocol was developed; the protocol is based on a first capturing step obtained with a hydrophobic interaction chromatography (HIC) (FIG. 6A); an intermediate step based on ion exchange chromatography (IEC) (FIG. 6B); a final polishing step carried out with gel filtration. All chromatographic steps were performed with the AKTA Prime system (GE Healthcare).

Hydrophobic Interaction Chromatography (HIC)

This step was performed with a HiTrap Octyl FF of 5 mL (GE Healthcare). At the beginning of the procedure, the column was equilibrated with one volume of loading buffer (50 mM sodium acetate, 350 mM NaCl and 100 mM ammonium sulphate, pH=5.5); before sample loading, a 3 M ammonium sulphate solution was added to a clarified extract to gain the final concentration of 100 mM ammonium sulphate and then the extract was filtered through a 0.2 μm filter (Millipore). The sample was applied to the column at 1 mL/min flow rate. The column was washed with three volumes of loading buffer and with 50 mM sodium acetate, pH 5.5 until a flat baseline. Elution was carried out with 66% ethylene glycol in 50 mM sodium acetate. At the end of the procedure, the column was washed and regenerated with 20% ethanol.

Ion Exchange Chromatography (IEC)

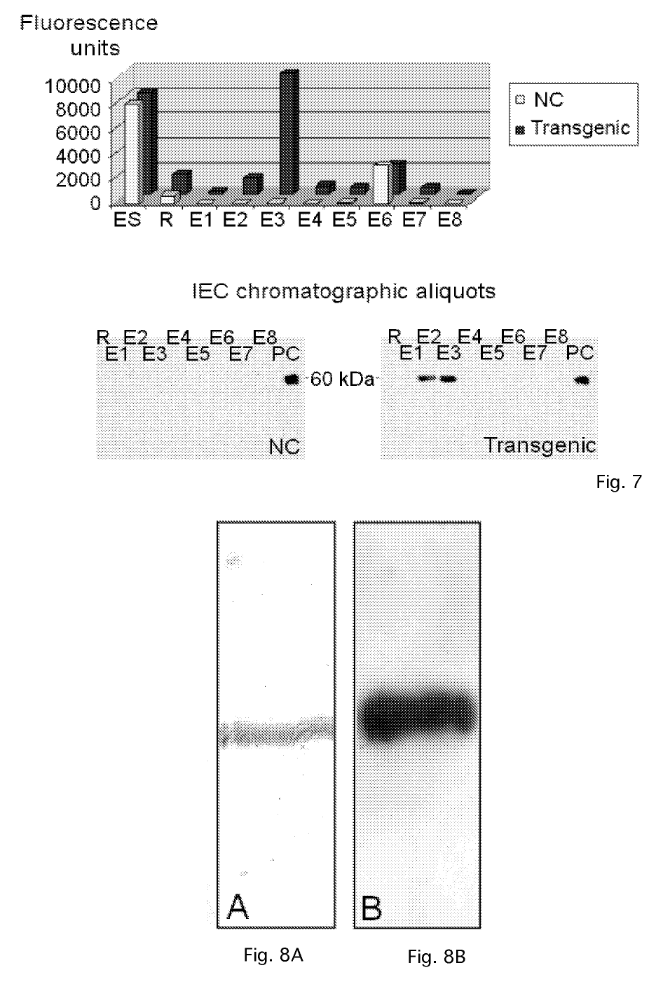

For IEC, a HiTrap SP FF of 5 mL (GE Healthcare) containing a cationic resin was used. The column was equilibrated with 50 mM sodium acetate (soln. A); then, the HIC eluted fraction, diluted 1:1 with the same buffer, was loaded. After column washing, a discontinuous gradient elution was performed using increasing amounts of NaCl equal to 15, 20 and 100% of a 1 M solution in soln. A. At the end, the column was regenerated with 20% ethanol. Immunologic assays carried out on aliquots collected from each chromatographic operation demonstrated that recombinant human acid beta-glucosidase is eluted with the solution containing 20% of NaCl. Enzymatic activity tests performed on eluted fractions obtained from protein extracts of non-transformed seed showed that the separation of human acid beta-glucosidase from the endogenous component responsible for a GCase-like activity occurs in this chromatographic step. In particular, it was established that at 20% NaCl concentration the endogenous component is efficiently retained in the column (FIG. 7).

Gel Filtration

For gel filtration, a HiPrep 16/60 Sephacryl S-100 High Resolution column (GE Healthcare) and a elution buffer composed of 20 mM sodium acetate and 200 mM NaCl, pH 5.5 were used. The column was initially washed with two volumes of the buffer, then the IEC eluted product was loaded at a 0.3 mL/min flow rate. The peak of interest was analyzed by SDS-PAGE and Western blotting (FIGS. 8A and 8B).

### Example 7

### Determination of GCase Enzymatic Activity

Recombinant human GCase activity was assayed using 4-MUG (4-methylumbelliferyl (β-D-glucoside, Sigma) as substrate. The reaction mixture contained 75 mM potassium phosphate buffer pH 5.9, 0.125% w/v taurocholate and 3 mM 4-MUG. The reaction was carried out at 37° C. for 1 h, using 10 μL of sample in 300 μL of assay solution. The reaction was stopped adding 1690 μL of 0.1 M glycine-NaOH, pH 10.0. The enzymatic activity was measured with a fluorimeter at an excitation wavelength of 365±7 nm and an emission wavelength equal to 460±15 nm. One unit (U) was defined as the amount of enzyme releasing one micromole of substrate per minute. Different sample quantities were tested in comparison with known amounts of commercial imiglucerase. The fluorimetric assay demonstrated that recombinant human GCase produced in rice endosperm is active and characterized by the same reaction kinetic of commercial imiglucerase.

### Example 8

### Determination of the N-Terminal Sequence of Recombinant GCase

The correctness of GCase N-terminal sequence was ascertained by protein microsequencing. For this purpose, an enzyme aliquot was purified with HIC and IEC, loaded in a SDS-polyacrylamide gel and, at the end of electrophoresis, transferred to a PVDF membrane using the Trans-Blot Semi-Dry apparatus (transfer conditions: 25 V for 30 minutes in 10 mM CAPS buffer and 10% methanol, pH 11.0). After the transfer, the membrane was stained with 0.25% (w/v) Coomassie-blue R-250 solution in 50% methanol for 5 minutes, washed with water and destained with a 50% methanol solution for 10 minutes to visualize the band corresponding to the protein of interest. Microsequencing was carried out according to the Edman degradation procedure (Edman, 1950). The analysis showed the presence of a nonapeptide (ARPCIPKSF) perfectly overlapping with the N-terminal sequence of the mature form of human acid beta-glucosidase. Therefore, it can be concluded that the rice glutelin 4 signal peptide is well recognized by the ER membrane system and correctly removed during the internalization process.

### Example 9

### MALDI-TOF Analysis on Purified GCase

Protein Digestion with Trypsin

After gel electrophoresis and staining with 0.25% (w/v) Coomassie-Blue R-250 water solution, 50% methanol, 10% glacial acetic acid, the protein band corresponding to recombinant GCase was cut and washed at 37° C. with 300 μL of 100 mM NH₄HCO₃ and 100% acetonytrile (ACN) (50:50 v/v) solution, pestled and dehydrated with further 100 μL of ACN. The protein band was subsequently treated with 50 μL of 20 mM DTT in 100 mM NH₄HCO₃ at 56° C. for 1 hour to obtain a reduction of disulfide bridges and alkylated with 50 μL of 50 mM IAA (iodoacetamide) in 100 mM NH₄HCO₃ for 30 minutes. Furthermore, the protein band was washed with 300 μl of 100 mM NH₄HCO₃, then with 300 μL of 20 mM NH₄HCO₃ and 100% ACN (50:50 v/v) solution and dehydrated again with the addition of 100 μL ACN. Finally, the protein band was rehydrated with 5-10 μL of digestion buffer containing 100 mM NH₄HCO₃ and 50 ng/μL trypsin (Promega); after 30 minutes, 20 μL of 20 mM NH₄HCO₃ were added. After sample incubation at 37° C. overnight, the buffer containing the tryptic peptides was removed and a further peptide extraction was performed by adding 10 μL of 2% formic acid and 60% ACN (50:50 v/v) solution to the sample. The two extracts were pooled together and used for MALDI-TOF analyses (Perkin Elmer).

Peptide Purification by C18 Resin

The tryptic digest was purified and desalted with a C18 zip-tip (Millipore). Tips were washed four times with 10 μL of 100% ACN and three times with 10 μL of 0.1% TFA (trifluoroacetic acid). The sample was then added to the activated tips; after washes with 0.1% TFA, the peptides bound to the inverse phase resin of C18 zip-tip were eluted with 10 μL of 100% ACN and 0.1% TFA at a ratio 70:30 (v/v).

Protein Identification by Peptide Mass Fingerprinting in MALDI-TOF Mass Spectrometry

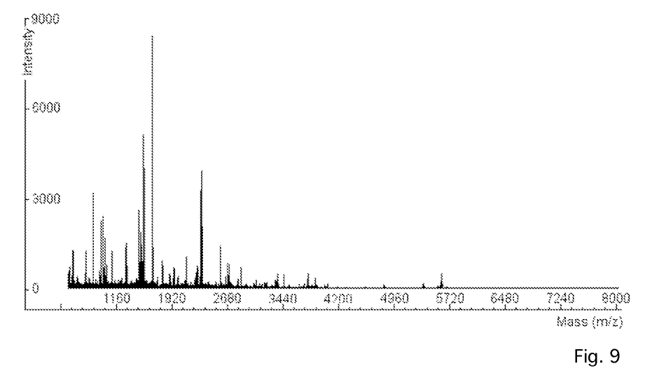

Sample preparation for MALDI-TOF analyses was performed by adding 1 μL of CHCA resin (α-ciano-4-hydroxycinnamic acid) concentrate solution placed on a metallic support to 1 μL of purified peptides. The analysis (FIG. 9) demonstrated that the protein purified with the procedure described in example 6 surely corresponds to the human acid beta-glucosidase. In particular, its identification was obtained with a score of 10⁻³² and a sequence coverage with recognized peptides equal to 53%. These results are of absolute guarantee, considering that the level of significance is reached with a score ≦10⁻⁶ and a percent coverage ≧30%. Interestingly, both the N-terminus and the C-terminus of the protein were found among the peptides recognized in MALDI-TOF (see table 1 for the complete list).

TABLE 1
Tab. 1: main assignations of the tryptic peptides
analyzed by MALDI-TOF mass spectrometry

| Mteorical | ΔM | assignation |
|---|---|---|
| 840.464 | −0.010 | 1-7 N-terminus |
| 883.451 | +0.104 | 322-329 |
| 932.472 | +0.122 | 426-433 |
| 950.461 | +0.102 | 347-353 |
| 976.586 | +0.073 | 286-293 |
| 988.655 | +0.045 | 156-163 |
| 1002.517 | +0.084 | 278-285 |
| 1086.628 | +0.209 | 464-473 |
| 1281.585 | +0.008 | 121-131 |
| 1459.792 | −0.023 | 396-408 |
| 1527.722 | +0.006 | 199-211 |
| 1630.818 | +0.995 | 263-277 |
| 1646.794 | +0.018 | 107-120 |
| 1664.806 | +0.038 | 347-359 |
| 1714.937 | −0.087 | 426-441 |
| 1870.896 | +0.120 | 330-346 |
| 2099.099 | +0.170 | 304-321 |
| 2304.190 | +0.074 | 442-463 |
| 2562.432 | +0.205 | 164-186 |
| 2846.256 | +1.025 | 132-155 |
| 3087.435 | +1.005 | 132-157 |
| 3139.530 | +0.325 | 199-224 |
| 3217.641 | +1.521 | 258-285 |
| 3424.784 | +1.007 | 506-535 C-terminus |

### Example 10

### Production of the GAA Expression Vector

This example describes a method for the endosperm-specific expression of human acid alpha-glucosidase in rice. In particular, the realization of the final expression vector pSV2006[GluB4pro/LLTCK/GAA/NOSter] is reported. This vector was realized by replacing the GCase gene with the GAA gene in the previous-mentioned pSV2006[GluB4pro/LLTCK/PSGluB4/GCase/NOSter] vector.

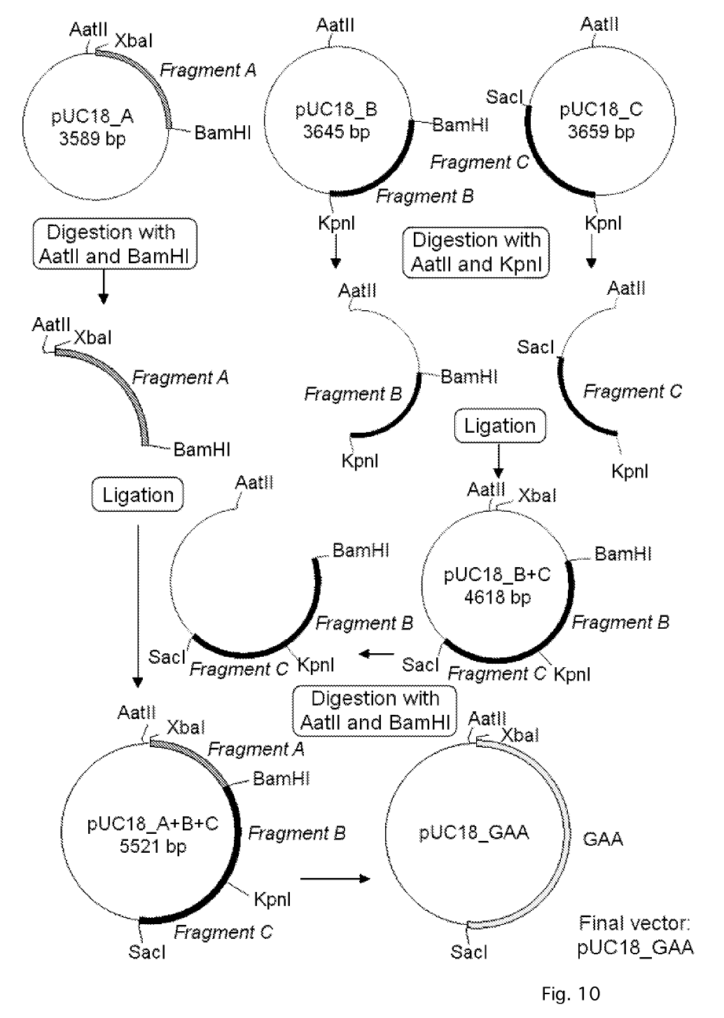

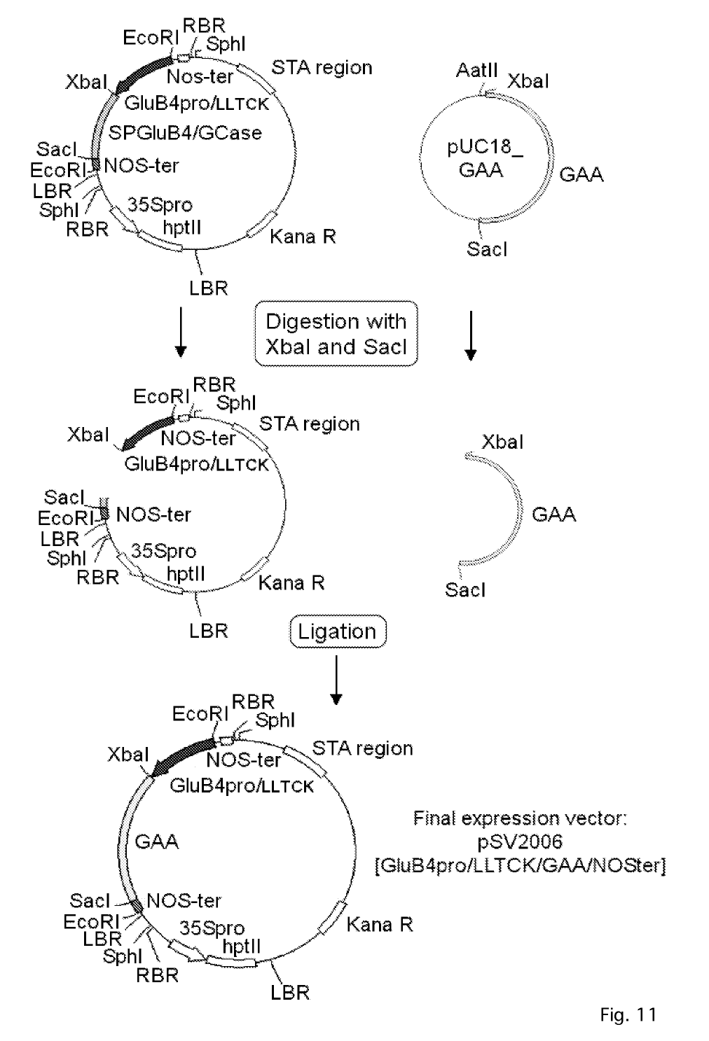

The coding sequence of the human acid alpha-glucosidase (GenBank Acc. No NM_{—}000152) was modified in order to increase transgene expression levels in rice endosperm; the new GAA coding sequence was rewritten on the basis of rice codon usage. Furthermore, it was decided to replace the native GAA signal peptide with PSGluB4, i.e. the same transit peptide used to target recombinant GCase in the ER lumen. Since the GAA coding sequence is 2850 bp long, it was artificially synthesized in three fragments (A, B and C). In order to assemble these fragments in a clearly oriented fashion, specific enzyme restriction sites were introduced by synonymous point mutation at their edges. A Xba I and Sac I site was introduced respectively at the 5′ end of the first fragment and at the 3′ end of the third fragment to ease cloning of the whole GAA gene into pSV2006. The assembly of the three GAA fragments was performed in the pUC18 vector (FIG. 10); after having checked its whole sequence (SEQ ID No: 9), the gene was excised from pUC18 by digestion with Xba I and Sac I and cloned in substitution of the GCase gene within pSV2006[GluB4pro/LLTCK/PSGluB4/GCase/NOSter], to give the final expression vector pSV2006[GluB4pro/LLTCK/PSGluB4/GAA/NOSter] (FIG. 11).

### Example 11

### Western Blotting on GAA Protein Extracts

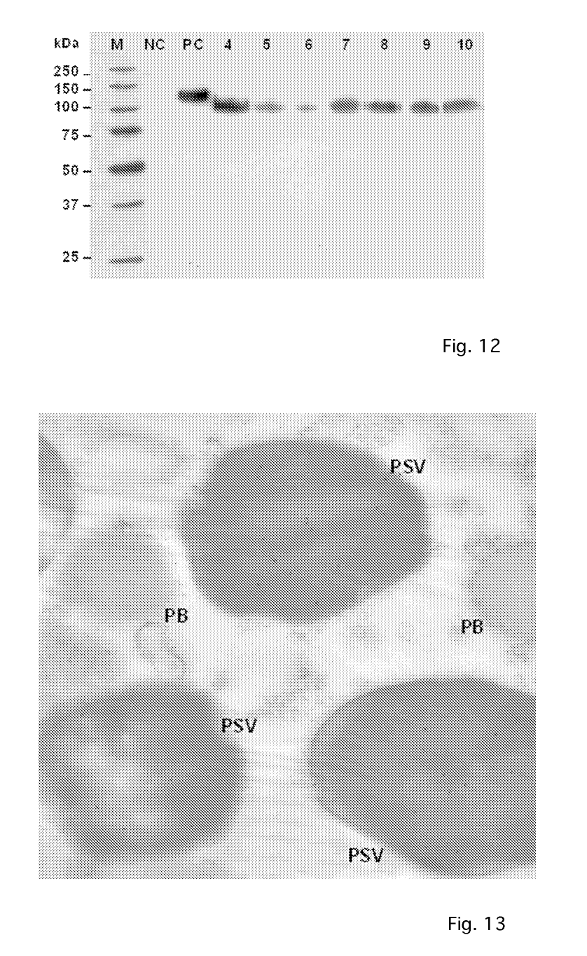

Five dehulled seeds were ground with 1 mL of 350 mM NaCl in 50 mM sodium phosphate buffer (pH=6.2) using mortar and pestle. The resulting homogenate was incubated on ice for 1 h under agitation and then centrifuged at 15000×g for 45 min at 4° C. The supernatant (20 μg soluble protein) was loaded in a 10% polyacrylamide gel together with Precision Protein Standard (BioRad). The gel was electroblotted to a 0.2 μm PVDF membrane (Millipore) with the Trans-blot SD apparatus (BioRad). The blot was blocked with 7.5% non-fat dry milk in PBS buffer (Oxoid) for 1 h at room temperature. After washing, the primary rabbit polyclonal antibody, produced using lyophilized alpha alglucosidase (Myozyme™, Genzyme Corp.) as antigen, was diluted 1:5000 in the blocking buffer and the blot was incubated for 1 h at room temperature. Then, the HRP-conjugated secondary antibody (Sigma Aldrich) was diluted 1:10000 and the membrane incubated for 1 h at room temperature. After the final washes, chemiluminescence was developed with ECL plus (GE Healthcare Bio-Sciences) (FIG. 12).

### Example 12

### Immunolocalization of Recombinant GAA in Rice Endosperm

The procedure was quite similar to that described for rice seeds transformed with the GCase construct.

Briefly, approximately 10-15 days after flowering, immature rice seeds were dehydrated and embedded in LR White Resin (London Resin Co. Ltd., Hamshire, UK). Blocks were polymerized at 60° C. for 24 h. Ultra-thin sections were cut with the ultramicrotome LKB Nova (Reichter) and mounted on nickel grids (Electron Microscopy Sciences) for immunolocalization. Sections were incubated with Normal Goat Antiserum (Aurion) diluted 1:30 in buffer C for 15 min and then with the anti-GAA serum (the same used for Western blot analyses) diluted 1:100 in buffer C for 1.5 h at room temperature. After washing, the sections were incubated for 1 h with a solution of goat anti-rabbit antibody conjugated with 15 nm colloidal gold (Aurion) diluted 1:40 in 0.02 M Tris-HCl pH 8.2 containing 0.9% NaCl and 1% BSA. Sections were stained with 0.1% lead citrate (Reynolds, 1963) and examined with a Philips CM10 transmission electron microscope (TEM). The same procedure was carried out on a sample derived from non-transformed rice.

As observed in sections of GCase seed, the immunogold labelling of GAA seed endosperm showed that the recombinant enzyme is specifically localized within protein storage vacuoles (PSVs) (FIG. 13). No signals were ever detected in protein bodies (PBs) or in the CR W3 negative control.

### Example 13

### ELISA on GAA Protein Extracts

Before performing ELISA, 2 ml of the anti-GAA antiserum antibody, produced in rabbit as mentioned in example 11, were purified by a Hitrap rProtein A FF column of 1 mL (GE Healthcare). Then the purified IgGs were conjugated to horseradish peroxidase (HRP) using the EZ-Link® Maleimide activated Horseradish peroxidase kit (Pierce) as reported in the following protocol: 100 μL of Maleimide conjugation buffer were added to the 6-mg vial of 2-MEA; the solution was added to the IgG sample and the mixture incubated for 90 minutes at 37° C. After equilibration at room temperature, the IgG/2-MEA solution was applied to a desalting column, pre-equilibrated with 30 mL of Maleimide conjugation buffer. Subsequently, Maleimide conjugation buffer was added to the column and fractions of 0.5 mL were collected. To locate the protein peak, the absorbance of each fraction was read at 280 nm; the fractions containing the reduced IgG were pooled and added to the vial of activated HRP. The reaction was incubated for 1 hour at room temperature. Finally, a gel filtration using a superdex 200 10/300 GL column in Maleimide coating buffer (containing PBS and EDTA) was performed. The eluted peak was concentrated by Amicon Ultra-10 (Millipore) till a concentration of 0.85 μg/μL. The quality of the HRP-conjugated anti-GAA antibody was tested by ELISA. For this purpose, 1 mg/mL of antigen Myozyme was coated on a plate; after blocking, the conjugated was added in different dilutions and incubated at 37° C. for 30 minutes. The detection was performed using TMB substrate (3, 3′, 5, 5″-tetramethylbenzidine); the lowest detection limit of the antigen was obtained with a 1:1000 dilution of the HRP-conjugated anti-GAA antibody.

This antibody was then used to perform a sandwich ELISA on crude protein extracts as described below.

The coating was performed by adding 100 μL of 15 ng/μL purified anti-GAA antibody in microwells of the ELISA plate and incubating at 4° C. overnight.

After blocking with 3% BSA in PBS for 1 hour at room temperature and a rinse with PBS 0.1% Tween-20, total protein seed extracts (diluted 1:10 or 1:100 in PBS, 0.1% Tween-20 and 1% BSA) were added and incubated for 30 minutes at 37° C. At the end of incubation, three washes were done and then the HRP-conjugated anti-GAA antibody was added to a dilution of 1:40 in PBS, 0.1% Tween-20 and 1% BSA and incubated for 30 minutes at 37° C. After four washes, the detection was performed using TMB substrate.

On the basis of ELISA tests carried out with known amounts of standard Myozyme, seed protein samples obtained from GAA primary transformants showed an average GAA content equal to 0.5% of total soluble proteins.

It is clear that modifications and/or additions of parts or steps may be made to method for the production of human recombinant lysosomal enzymes in a cereal endosperm as described heretofore, without departing from the scope of the present invention.

It is also clear that, although the present invention has been described with reference to some specific examples, a person of skill in the art shall certainly be able to achieve many other equivalent forms of method for the production of human recombinant lysosomal enzymes in a cereal endosperm having the characteristics as set forth in the claims and hence all coming within the field of protection defined thereby.

1. Method for the production of recombinant human lysosomal enzymes, suitable for therapeutic use, in a cereal endosperm, comprising:
the construction of a plant expression vector for the transformation of such cereal, containing a nucleotide sequence harbouring the following elements:
i) the rice glutelin 4 promoter (GluB4pro) as an endosperm-specific promoter upstream the gene encoding said lysosomal enzyme;
ii) the leader known as LLTCK as a 5′ UTR region;
iii) the PSGluB4 sequence encoding a signal peptide used in rice to target the precursor of glutelin 4 inside the endoplasmic reticulum, said signal peptide being suitable to carry out a co-translational transfer of the newly synthesized lysosomal enzyme into the lumen of the endoplasmic reticulum of the endosperm cells and to determine the accumulation of said lysosomal enzyme in a specific cell compartment;
iv) a nucleotide sequence encoding the mature form of the human lysosomal enzyme;
v) a 3′ UTR of natural or artificial origin;
a step of cereal plant transformation using said vector, whereby the lysosomal enzyme is obtained and confined in an endosperm, which is not eventually absorbed by the embryo, and the presence of large quantities of the lysosomal enzyme in the endosperm does not negatively affect seed viability and germination speed;
a step of lysosomal enzyme accumulation inside the seed endosperm of said cereal plant.

2. Method as in claim 1, wherein said method provides lysosomal enzyme accumulation within endosperm protein storage vacuoles (PSVs) or protein bodies (PBs).

3. (canceled)

4. Method as in claim 1, wherein the nucleotide sequence of the expression vector is as indicated in SEQ ID No: 1.

5. Method as in claim 1, wherein the expression vector is introduced in bacterial strains, which are, directly or indirectly, used for plant transformation.

6. Method as in claim 5, wherein the bacterial strain chosen belongs to a group which comprises Escherichia coli, Agrobacterium tumefaciens and Agrobacterium rhizogenes.

7. (canceled)

8. Method as in claim 5, wherein the bacterial strain is used for the transformation of embryonic calli of rice (Oryza sativa ssp. japonica, var. CR W3).

9. Method as claim 1, wherein the lysosomal enzyme is the human acid beta-glucosidase.

10.-11. (canceled)

12. Method as in claim 54, wherein the industrial manufacturing process submits the mature seeds harvested from transformed cereal plants to dehulling and whitening operations in order to eliminate the fibrous component, the germ and the aleuronic layer containing protein contaminants.

13. Method as in claim 1, comprising a step of purification of the recombinant lysosomal enzyme, which comprises a hydrophobic interaction chromatography, a ion exchange chromatography and a gel filtration.

14. (canceled)

15. Method as in claim 13, wherein the purification step comprises the application of chromatographic resins that have similar chemical compositions and/or structure and/or functions, the partially modification of elution conditions, the duplication of a passage.

16. Nucleotide sequence suitable in plant transformation for the expression of recombinant human lysosomal enzymes, suitable for therapeutic use, in a cereal endosperm, comprising the following elements:
i) the rice glutelin 4 promoter (GluB4pro) as an endosperm-specific promoter upstream the gene encoding said lysosomal enzyme;
ii) the leader known as LLTCK as a 5′ UTR region;
iii) the PSGluB4 sequence encoding a signal peptide used in rice to target the precursor of glutelin 4 inside the endoplasmic reticulum, said signal peptide being suitable to carry out a co-translational transfer of the newly synthesized lysosomal enzyme into the lumen of the endoplasmic reticulum of the endosperm cells and to determine the accumulation of said lysosomal enzyme in a specific cell compartment;
iv) a nucleotide sequence of natural or artificial origin encoding the mature form of the human lysosomal enzyme;
v) a 3′ UTR of natural or artificial origin.

17.-19. (canceled)

20. Sequence as in claim 16, wherein the nucleotide sequence of the iv) element is the sequence encoding the mature form of human acid beta-glucosidase or the sequence encoding the mature form of human acid alpha-glucosidase.

21. Sequence as in claim 16, wherein, the 3′ UTR of the v) element is the NOS terminator or the terminator of the GluB4 gene.

22. Nucleotide sequence as in claim 16, as indicated in SEQ ID No: 1.

23. (canceled)

24. Sequence complementary to the nucleotide sequences as in claim 16 or deriving from mutation events, like deletions, insertions, substitutions of one or more nucleotides in sequences as in claim 16, or their complementary sequences.

25.-29. (canceled)

30. Molecular vector for the expression of human recombinant lysosomal enzymes suitable for therapeutic use, in a cereal endosperm, comprising the nucleotide sequence as in claim 16.

31. Vector as in claim 30, wherein the lysosomal enzyme is the human acid beta-glucosidase or the human acid alpha-glucosidase.

32.-33. (canceled)

34. Use of the expression vector as in claim 30 for the transformation of a cereal for the production of human lysosomal enzymes suitable for therapeutic use.

35. (canceled)

36. Bacterial strain harbouring vectors as in claim 30, chosen in a group comprising the species Escherichia coli, Agrobacterium tumefaciens and Agrobacterium rhizogenes.

37. Cereal plant cells transformed with an expression vector as in claim 30.

38. (canceled)

39. Cells as in claim 37 belonging to the cultivated rice species (Oryza sativa L.).

40. Cells as in claim 37 belonging to the Graminaceae family (Poaceae) like for example mayze (Zea mays L.), barley (Hordeum vulgare L.) and wheat (Triticum spp.).

41. Seed of the transformed cereal plant for the expression of human lysosomal enzymes suitable for therapeutic use, containing an expression cassette derived from a vector as in 30.

42. (canceled)

43. Seed as in claim 41, wherein the transformed cereal plant belongs to cultivated rice species (Oryza sativa L.).

44. Transformed cereal plant for the expression of human lysosomal enzymes suitable for therapeutic use, transformed by an expression vector as in claim 30.

45. (canceled)

46. Transformed cereal plant as in claim 44 belonging to the cultivated rice species (Oryza sativa L.).

47. Progenies obtained by self-fertilization, natural or artificial crossing, or transformed lines selected from a transformed plant as in claim 44.

48.-50. (canceled)

51. Seed as in claim 41 for use in enzyme replacement therapy.

52. Seed as in claim 51, for use in enzyme replacement therapy of the following diseases: Gaucher disease, Glycogenosis type II, Fabry disease, Niemann Pick B disease, Mucopolysaccharidoses I, II, IV.

53. (canceled)

54. Method as in claim 1, comprising a step of cereal seed industrial manufacturing.
